(19) United States
(12) Patent Application Publication
(10) Pub. No.: US 2002/0123665 A1
(43) Pub. Date: Sep. 5, 2002

(54) Medical scope having sheath made from superelastic material

(75) Inventor: Gary H. Miller, Milpitas, CA (US)

Correspondence Address: Ralph W. Selitto, Jr., P.O. Box 1477, Edison, NJ, 08818-1477, US

(73) Assignee: Ethicon, Inc.

(21) Appl. No.: 09/752,259

(22) Filed: Dec. 29, 2000

(51) Int. Cl. A61B 1/00

(52) U.S. Cl. 600/143

(57) ABSTRACT

A medical scope adapted for use in viewing an image of an interior section of a body includes a housing having a viewing mechanism for viewing the image. An elongated shaft extends from the housing and includes a sheath and an image transmitting mechanism extending through the sheath for optically transmitting the image from the interior section to the viewing mechanism. The sheath is made from a superelastic material such that the sheath has an elasticity greater than that of the image transmitting mechanism, whereby the bending of the shaft and hence the image transmitting mechanism is restricted primarily by the elastic limit of the image transmitting mechanism and not by the elastic limit of the sheath. The medical scope of the present invention can be any type of medical scopes, including rigid, semi-flexible and flexible endoscopes, medical telescopes, hysteroscopes, bronchialscopes and cystoscopes.

### FIELD OF THE INVENTION

The present invention relates to a medical scope and, more particularly, to a medical scope having a sheath made from a superelastic material. As used herein, the term “medical scope” shall mean a scope adapted for use in the medical field for viewing an interior section of a body, such as medical telescopes, hysteroscopes, bronchialscopes and cystoscopes.

### BACKGROUND OF THE INVENTION

In general, medical endoscopy telescopes are categorized into three groups in accordance with their physical characteristics: rigid endoscopes, semi-rigid endoscopes; and flexible endoscopes. Rigid telescopes typically have sizes (i.e., diameters) as small as 2.7 mm and as large as 10 mm, while semi-rigid telescopes typically have smaller sizes, varying in diameter from about 1.6 mm to about 2.7 mm. Flexible telescopes typically range, in diameter, from about 0.5 mm to about 8 mm.

A conventional semi-rigid (also known as “semi-flexible”) imaging telescope includes imaging and illumination fiber optic bundles having from about five thousand to about seventy thousand optical fibers or strands. The fiber optic bundles are covered by a sheath such that they are protected therewithin and such that they are prevented from over-bending during the use of the telescope. The sheath is typically made from a high tensile strength material, such as stainless steel, and hence are not highly elastic (i.e., not as elastic as the optical strands carried therethrough).

In general, the image resolution of the telescope described above is enhanced by increasing the number of optical strands carried therethrough. As the number of optical strands increases, the telescope becomes less flexible, and its outer diameter becomes larger. For maximizing the imaging performance of the telescope (e.g., image resolution, clarity, linearity, hue and percent transmission), the telescope is provided with the largest possible number of optical strands, while maintaining its outer diameter and hence the sheath thickness to a minimum. The minimum bend radius (i.e., the minimum radius at which the telescope is bent without being damaged) of the telescope is dependent primarily upon the wall weight (i.e., wall thickness) and the outer diameter of the sheath. Because the wall weight of the sheath is minimized for any given outer diameter, the protection provided by the sheath to the optical strands carried therethrough is rather limited. For instance, if the telescope is flexed to a bend radius such that the fiber optic bundles are under-stressed but the sheath is over-stressed, the sheath can reach its elastic limit, kink and cause damage to the fiber optic bundles, rendering the telescope inoperable. In other words, the extent to which the telescope can be bent without being damaged is significantly restricted by the sheath.

Superelastic/shape memory materials have been utilized in various medical devices in the past (see, for instance, U.S. Pat. Nos. 4,969,709; 5,193,263; and 5,531,664). However, it is believed that none of these devices specifically addresses the problems discussed above.

U.S. Pat. No. 5,607,435 discloses a medical instrument for endoscopic-type procedures. More particularly, the instrument includes a tubular section and bundles of optical fibers extending therethrough. In use, the tubular section is fed through a delivery tube in order to deliver same to a desired location within a body. While the tubular section has a wall made from a superelastic material, this patent does not address the problems discussed above.

### SUMMARY OF THE INVENTION

The present invention overcomes the disadvantages and shortcomings of the prior art discussed above by providing a new and improved medical scope adapted for use for viewing an image of an interior section of a body. More particularly, the scope includes a housing having a viewing mechanism for viewing an image of an interior section of a body. An elongated shaft extends from the housing and includes a sheath and an image transmitting mechanism extending through the sheath for optically transmitting an image from an interior section of a body to the viewing mechanism. The sheath is made from a superelastic material such that it has an elasticity greater than that of the image transmitting mechanism, whereby the maximum bending of the shaft and hence the image transmitting mechanism is restricted primarily by the elastic limit of the image transmitting mechanism and not by the elastic limit of the sheath. The present invention can be used in connection with any type of medical scopes, including rigid, semi-flexible and flexible endoscopes, medical telescopes, hysteroscopes, bronchialscopes and cystoscopes.

### BRIEF DESCRIPTION OF THE DRAWINGS

For a more complete understanding of the present invention, reference is made to the following detailed description of an exemplary embodiment considered in conjunction with the accompanying drawings, in which:

FIG. 1 is a schematic view of an endoscope constructed in accordance with the present invention;

FIG. 2 is a schematic, cross-sectional view of an elongated shaft of the endoscope shown in FIG. 1;

FIG. 3 is a modified view of a distal section of the endoscope shown in FIG. 1; and

FIGS. 4 and 5 are views showing fiber optic bundle configurations different from that shown in FIG. 2.

### DETAILED DESCRIPTION OF THE EXEMPLARY EMBODIMENT

Although the present invention can be used in conjunction with any type of medical scopes, it is particularly suitable for use in connection with a semi-rigid endoscope. Accordingly, the present invention will be described hereinafter in connection with such an endoscope. It should be understood, however, that the following description is only meant to be illustrative of the present invention and is not meant to limit the scope of the present invention, which has applicability to other types of medical scopes, such as medical telescopes, hysteroscopes, bronchialscopes and cystoscopes.

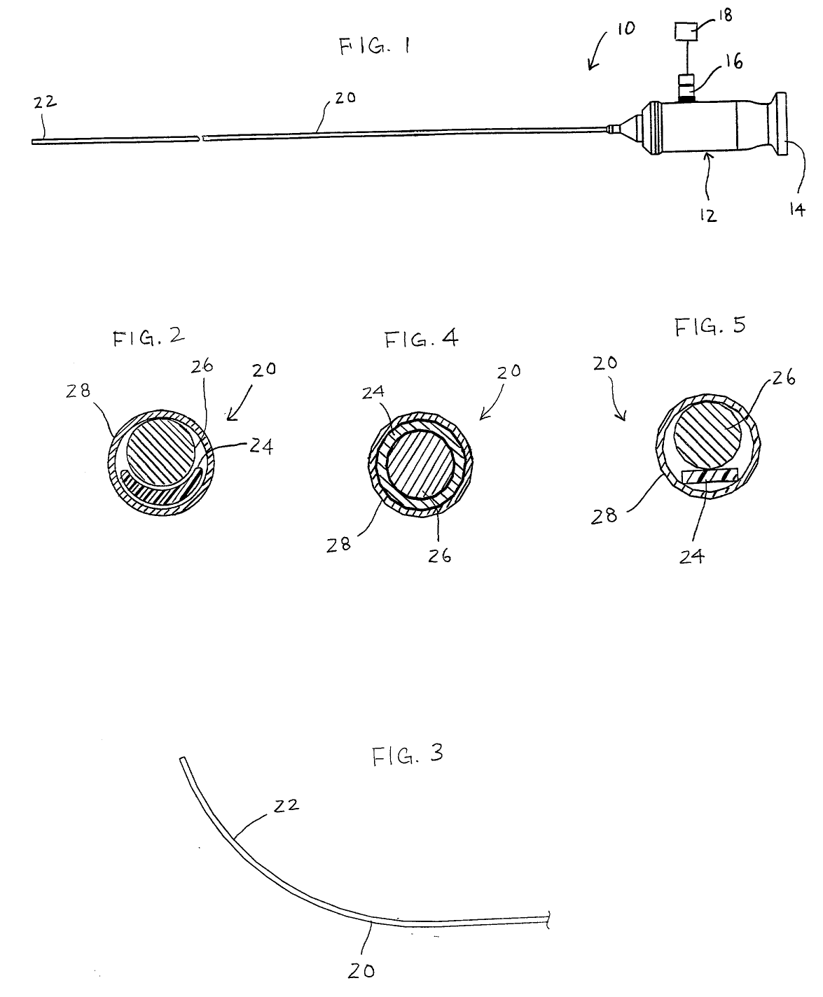

FIG. 1 illustrates a semi-rigid endoscope 10 constructed in accordance with the present invention. The endoscope 10 includes a housing 12 (i.e., a mounting crown or adapter) which has a construction and operation similar to those of housings of conventional imaging endoscopes. For instance, the housing 12 includes an eyepiece 14 for viewing an image therethrough and a light coupling 16 for allowing an external light source 18 to be coupled to the housing 12. The endoscope 10 is also provided with an elongated shaft 20 (i.e., tube) projecting from an end of the housing 12 opposite the eyepiece 14 and having a distal end 22 remote from the housing 12. Fiber optic bundles 24, 26 (see FIG. 2) extend through the elongated shaft 20 for carrying illumination light and images, respectively, therethrough. The fiber optic bundles 24, 26 include optical strands or fibers, the number of which is determined by various requirements considered in making conventional endoscopes. For instance, the fiber optic bundles 24, 26 can be provided with from about five hundred to about seventy thousand optical strands.

Referring to FIGS. 1 and 2, a sheath 28 is provided for covering and/or housing the fiber optic bundles 24, 26. More particularly, the sheath 28 is made entirely from a superelastic/shape memory material. While the sheath 28 can be made from any superelastic/shape memory materials, it is preferably made from a nickel—titanium alloy (also known in the metallurgy field as “Nitinol”), such as materials available from Shape Memory Applications, Inc., San Jose, Calif., under its Manufacturer Lot No. T-697, Inventory Control No. SSST3519 and/or Raw Material Lot No. RM0956. Of various nickel—titanium alloys, a nickel—titanium alloy having the following properties is particularly suitable for use as a material for the sheath 28 of the present invention.

| | |
|---|---|
| Alloy Code | S |
| Condition | Straight Annealed |
| Surface | Centerless Ground OD/Oxide ID |
| Ingot Aₚ | −2° C. |
| Chemical Composition (Weight %) | Ni = 55.6; Ti = balance; C ≦ 0.05; |
| | and O ≦ 0.05 |

As is known in the art, superelastic/shape memory materials are materials that exhibit reversible, stress-induced martensite at a temperature above their austenitic finish temperature (A_{f}) . In other words, superelastic materials exhibit springy, “rubber-like” elasticity, while maintaining relatively high tensile strength. These materials also exhibit shape memory characteristics (i.e., the ability to recover their previous shape when they revert to austenite from martensite). In this regard, it should be noted that while the nickel—titanium alloy described above is particularly suitable for use as a material for the sheath 28, other types of superelastic/shape memory alloys, such as nickel—titanium—niobium (Ni—Ti—Nb) alloys and cooper (Cu) alloys, can be used in connection with the present invention. Accordingly, the nickel—titanium alloy described above is only meant to be illustrative of the present invention and is not meant to limit the scope of the present invention.

The sheath 28 of the endoscope 10 can be made by using any conventional processes for making micro-tubes from superelastic/shape memory materials. Moreover, the sheath 28 is sized and shaped such that the shaft 20 is self-supporting (i.e., the shaft 20 is adapted to be delivered to a desired location in a body without the use of a separate delivery tube during an endoscopic procedure). While the sizes (e.g., the inside and outside diameters and the thickness) and shape of the sheath 28 can vary depending upon various requirements normally considered in making conventional endoscopes (e.g., the number of optical strands), a sheath having an inside diameter ranging from about 0.016 inch to about 10 mm and a thickness ranging from about 0.0015 inch to about 0.05 inch is particularly suitable for use in connection with the endoscope 10 of the present invention. For instance, when a 30 K fiber optic bundle (i.e., an optical bundle having thirty thousand optical strands) is used, a sheath having an inside diameter of 0.068 inch, an outside diameter of 0.078 inch and a thickness of 0.005 inch is particularly suitable. The sheath 28 is assembled with the fiber optic bundles 24, 26 in a conventional manner. Likewise, the shaft 20 is connected to the housing 12 in a conventional manner.

It should be appreciated that because of the superelasticity of the sheath 28, the shaft 20 of the present invention can be flexed to a minimum bend radius much smaller than the one associated with a comparably sized conventional shaft made from stainless steel. More particularly, because the sheath 28 is made from a superelastic material, which has an elasticity greater than that of the optical strands of one or both of the fiber optic bundles 24, 26, the minimum bend radius associated with the endoscope 10 is dependent not upon the sheath 28 but upon the elastic limit of the optical strands. Moreover, because the shaft 20 is self-supporting (i.e., the shaft 20 can be delivered to a desired location in a body without the use of a separate delivery tube), the benefit of the superelastic sheath 28 is fully realized.

It should be noted that the present invention can have numerous modifications and variations. For instance, if the shaft 20 needs to have a predetermined shape for viewing in lateral, anterior and/or posterior directions, the superelastic material can be thermally processed in a conventional manner such that the predetermined shape is set in the sheath 28 and hence in the shaft 20, while maintaining its superelastic properties. By way of example, the distal end 22 of the shaft 20 can be set with a curved shape (see FIG. 3). In addition, rather than being made entirely from a superelastic/shape memory material, only preselected portions of the sheath 28 can be made from a superelastic/shape memory material. As is conventional in the medical scope field, the fiber optic bundles 24, 26 can be provided with configurations different from the round/crescent configuration shown in FIG. 2 (see, for instance, FIGS. 4 and 5). The endoscope 10 can also be equipped with other components utilized in conventional endoscopes, such as fluid valves, access cannulae and articulating wires. Moreover, the sheath 28 can be provided with a cross-sectional shape other than a circular shape (e.g., oval, rectangular, etc.) and/or can be corrugated. Further, while the present invention has been described above in conjunction with a semi-rigid endoscope, it can be used in connection with other types of medical scopes, including flexible endoscopes, rigid endoscopes, which are equipped with different light and/or image transmitting mechanisms (i.e., glass rods), medical telescopes, hysteroscope, bronchialscope, cystoscope, etc.

It will be understood that the embodiment described herein is merely exemplary and that a person skilled in the art may make many variations and modifications without departing from the spirit and scope of the invention. All such variations and modifications, including those discussed above, are intended to be included within the scope of the invention as defined in the appended claims.

I claim:

1. A medical scope adapted for use in viewing an image of an interior section of a body, comprising a housing including viewing means for viewing the image; an elongated shaft extending from said housing and including a sheath and image transmitting means extending through said sheath for optically transmitting the image to said viewing means, said sheath being made from a superelastic material such that said sheath has an elasticity greater than that of said image transmitting means, whereby the bending of said shaft and hence said image transmitting means is restricted primarily by the elastic limit of said image transmitting means and not by the elastic limit of said sheath.

2. The medical scope of claim 1, wherein said shaft is self-supporting, whereby a distal end of said shaft can be delivered to the interior section without the use of a separate delivery tube.

3. The medical scope of claim 2, wherein said sheath has an inside diameter ranging from about 0.016 inch to about 10 mm.

4. The medical scope of claim 3, wherein said sheath has a wall thickness ranging from about 0.0015 inch to about 0.05 inch.

5. The medical scope of claim 2, wherein said superelastic material includes a nickel—titanium alloy.

6. The medical scope of claim 5, wherein said superelastic material has shape memory characteristics.

7. The medical scope of claim 6, wherein said sheath has a predetermined shape set therein.

8. The medical scope of claim 7, wherein said sheath includes a distal end having a curved shape set therein.

9. The medical scope of claim 2, wherein said image transmitting means includes a bundle of optical strands.

10. The medical scope of claim 9, wherein said shaft includes light transmitting means extending through said sheath for transmitting light from an external light source to the interior section.

11. The medical scope of claim 10, wherein said light transmitting means includes a bundle of optical strands.

12. The medical scope of claim 11, wherein said bundle of optical strands of said image transmitting means includes from about five hundred to about seventy thousand optical strands.
